(19) United States
(12) Patent Application Publication
(10) Pub. No.: US 2015/0150498 A1
(43) Pub. Date: Jun. 4, 2015

(54) SLEEP STUDY

(71) Applicant: UNITED SCIENCES, LLC, ATLANTA, GA (US)

(72) Inventors: EOHAN GEORGE, ATLANTA, GA (US); KAROL HATZILIAS, ATLANTA, GA (US); MAYOOR PATEL, ATLANTA, GA (US); GOVINDA PINGALI, ATLANTA, GA (US); BRIAN POZGAY, ATLANTA, GA (US); JIM RAUBOLT, ATLANTA, GA (US); WESS ERIC SHARPE, VININGS, GA (US); JACOB THOMPSON, ATLANTA, GA (US)

(73) Assignee: UNITED SCIENCES, LLC, ATLANTA, GA (US)

(21) Appl. No.: 14/094,241

(22) Filed: Dec. 2, 2013

(51) Int. Cl. A61B 5/00

(52) U.S. Cl. CPC A61B 5/4815

(57) ABSTRACT

Method, systems, and products are provided for sleep study. Embodiments include receiving, by a sleep study module, one or more biometric values capable of indicating the existence of a sleep disorder, the biometric values derived from information regarding the sleep of a patient sensed by one or more sensors of an earpiece worn within an ear of a sleeping patient; and recording, by the sleep study module, the plurality of biometric values for evaluation of the patient's sleep.

### BACKGROUND

Sleep studies are tests that record the body activity during sleep. Sleep studies are helpful in identification of sleep disorders. Sleep studies often are conducted in sleep centers. Sleep studies in sleep centers are expensive, and it is difficult to get good data in a sleep study in a sleep center. A sleep patient is inherently uncomfortable with sleeping in a new environment, causing sleep difficulties in addition to the ones that brought the patient to the sleep center in the first place.

### SUMMARY

Methods and apparatus are described for sleep study. Embodiments include receiving, by a sleep study module, one or more biometric values capable of indicating the existence of a sleep disorder, the biometric values derived from information regarding the sleep of a patient sensed by one or more sensors of an earpiece worn within an ear of a sleeping patient; and recording, by the sleep study module, the plurality of biometric values for evaluation of the patient's sleep.

The foregoing and other objects, features and advantages of the invention will be apparent from the following more particular descriptions of exemplary embodiments of the invention as illustrated in the accompanying drawings wherein like reference numbers generally represent like parts of exemplary embodiments of the invention.

### BRIEF DESCRIPTION OF THE DRAWINGS

FIG. 1 sets forth a line drawing of an example system of apparatus for sleep study.

FIG. 2 sets forth a line drawing of a display of sensed information and biometric values.

FIG. 3 sets forth a flow chart illustrating an example method of administering a sleep disorder.

FIG. 4 sets forth a flow chart illustrating an example method of sleep study.

### DETAILED DESCRIPTION OF EXAMPLE EMBODIMENTS

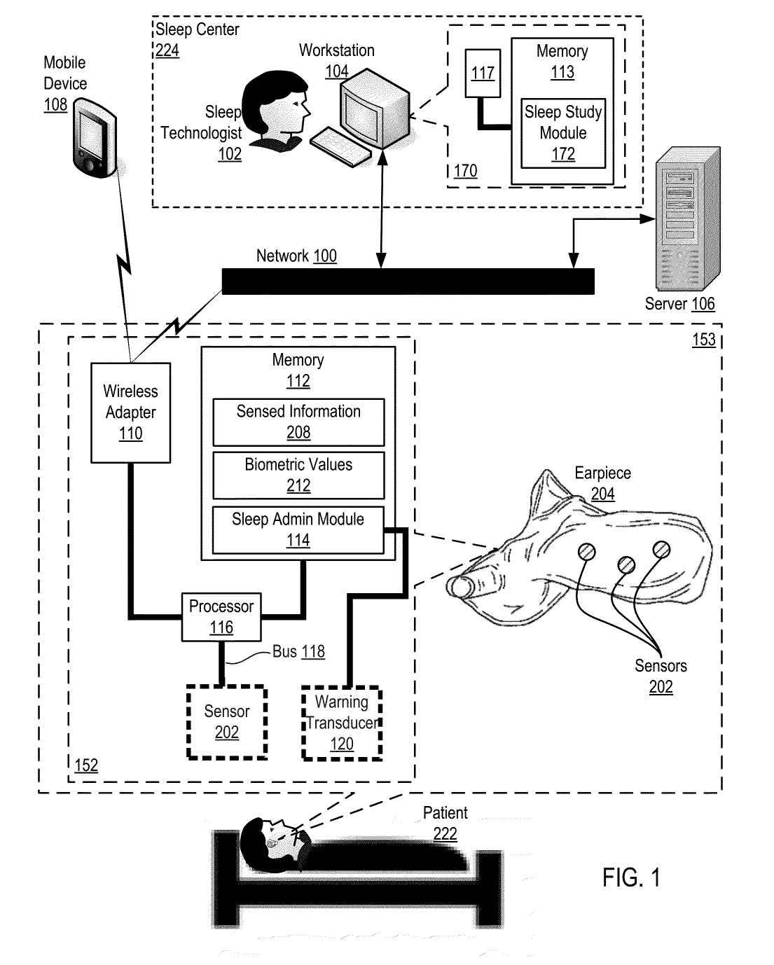

Example methods and apparatus or systems for sleep study are described with reference to the accompanying drawings, beginning with FIG. 1. FIG. 1 sets forth a line drawing of an example system of apparatus for sleep study. The example apparatus of FIG. 1 includes an earpiece (204). The earpiece has sensors (202) integrated into the earpiece, and the sensors are capable of sensing, when the earpiece is worn within an ear of a sleeping patient (222), information (208) regarding the sleep of the patient.

The earpiece (204) in this example is manufactured from a 3D image derived from an optical scan of the interior of the patient's ear canal. Creating a 3D image derived from an optical scan of the interior of the patient's ear canal can be carried out using methods and systems described in U.S. patent application Ser. Nos. 13/417,649; 13/417,767, 13/586,471;13/586.411; 13/586,459; 13/546,448; 13/586,448; 13/586,474; 14/040,973, 14/041,943; 14/049,666; 14/049,530; 14/049,687, all incorporated by reference herein in their entirety.

The example apparatus of FIG. 1 also includes a sleep administration module (114) operably coupled to the earpiece sensors (202). The sleep administration module is a module of automated computing machinery configured to receive the sensed information (208) from the sensors (202) and derive from the sensed information (208) one or more biometric values (212). The sleep administration module (114) is also configured to transmit, through a wireless data communications adapter (110) and a data communications network (100), to a sleep study module (172) in a sleep center (224), the biometric values (212) for evaluation. In the example of FIG. 1, the sensed information (208) can include electroencephalography, electromyography, electrooculography, electrocardiography, accelerometry, reflective pulse oximetry, audio, temperature, and other sensed information as may occur to those of skill in the art. Also in the example of FIG. 1, the biometric values (212) can include pulse rate, body temperature, blood oxygen level, rapid eye movement sleep, non-rapid eye movement sleep, snoring, blood pressure, muscle tension, and other values derived from sensed information as may occur to those of skill in the art.

Examples of sleep disorders amenable to study by the example apparatus of FIG. 1 include sleep hypopnea, sleep apnea, and a sleep disorder that is a precursor to an episode of sleep apnea. This paper tends to focus on apnea and hypopnea, but there are many more sleep disorders and related disorders amenable to sleep study, including, for example, epileptiform discharges, seizures, RBD, REM without atonia, multiple parasomnias (sleep terrors, sleep walking, sleep talking, catathrenia, exploding head syndrome, confusional arousals, hypnogogic hallucinations, hypnopompic hallucinations, sleep paralysis, etc . . . ), nocturnal movement disorders (bruxism, RLS, PLMD, muscle cramps, myoclonus, etc . . . ), multiple causes for sleep fragmentation (pain-related insomnia, excessive cortical and sub-cortical arousal, hyperhidrosis, etc . . . ), sleep-disordered breathing (all types including pediatric and adult), narcolpesy, cataplexy, delayed sleep phase, advanced sleep phase, idiopathic hypersomnolence, recurrent hypersomnolence, and day/night PSG testing also provides a host of other measures that are important such as EKG arrhythmias, peripheral O2/CO2 levels, respiratory drive via direct measures (RIP bands, intercostal EMG) and indirect measures (nasal airflow, air temperature flow, snoring), and periodic muscle analysis with EMG.

The sleep administration module (114) in the example of FIG. 1 is also configured to prevent an episode of sleep apnea through a warning implemented through a warning transducer (120) to the patient before an onset of sleep apnea when the sleep administration module determines that a precursor condition is present. Examples of warning transducers include a tone generator and speaker or earphone, a vibrator, a buzzer, or the like, integrated within the earpiece.

In the example of FIG. 1, the sleep administration module is disposed within an earpiece that is mounted in the patient's ear. In some embodiments, however, the sleep administration module is mounted in a mobile device (108), such as a smartphone, for example, and disposed within the patient's own residence where the patient routinely sleeps. In such embodiments, the patient can carry out the patient's portion of the work of a sleep study in the comfort of the patient's own home.

The system and apparatus in the example of FIG. 1 includes a computer processor (117) and computer memory (113) disposed within a sleep center (224) and coupled for data communications through a network (100) and adapter (110) to the earpiece (204) and its sensors (202). The computer memory (113) has disposed within it a sleep study module (172) that is a module of automated computing machinery that functions by receiving the biometric values (212) indicating whether a sleep disorder is present in the patient (222). These are the same biometric values derived from the sensed information (208) regarding the sleep of the patient. The sleep study module is also capable of making an automated determination whether the biometric values or the sensed information indicate that the patient has a sleep disorder and notifying the sleep technologist of any such determination.

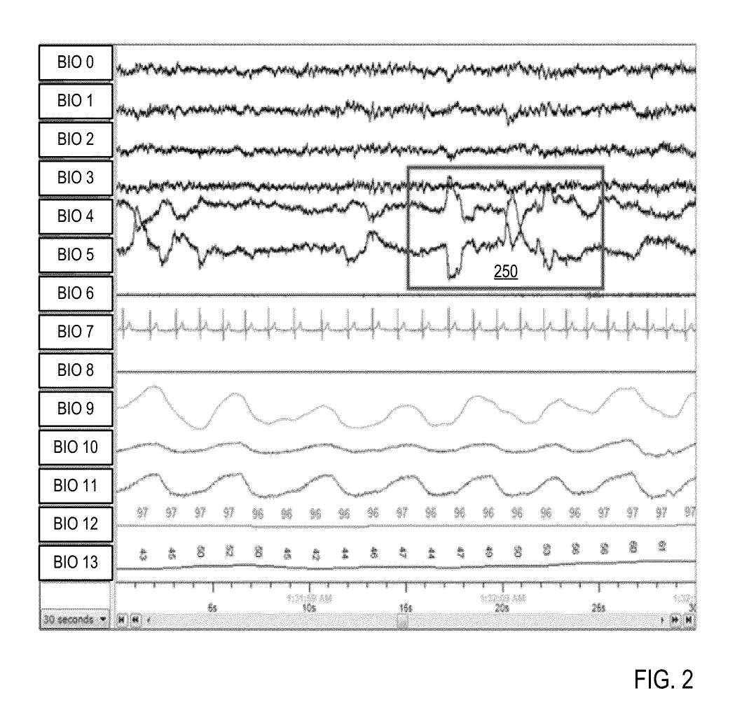

The sleep study module in this example also records the biometric values for use in evaluating attributes of the patient's sleep, and, as shown in FIG. 2, the sleep study module (172) displays the biometric values (212) and/or the sensed information (208) for review and analysis by a sleep technologist (102). FIG. 2 actually illustrates information and biometric values from a type of sleep study called polysomnography. The traces illustrated in FIG. 2 include EEG, EMG, and the like, including two EOG traces labeled BIO 4 and BIOS, which, in the portion of the traces inside the red box (250), indicate rapid eye movement.

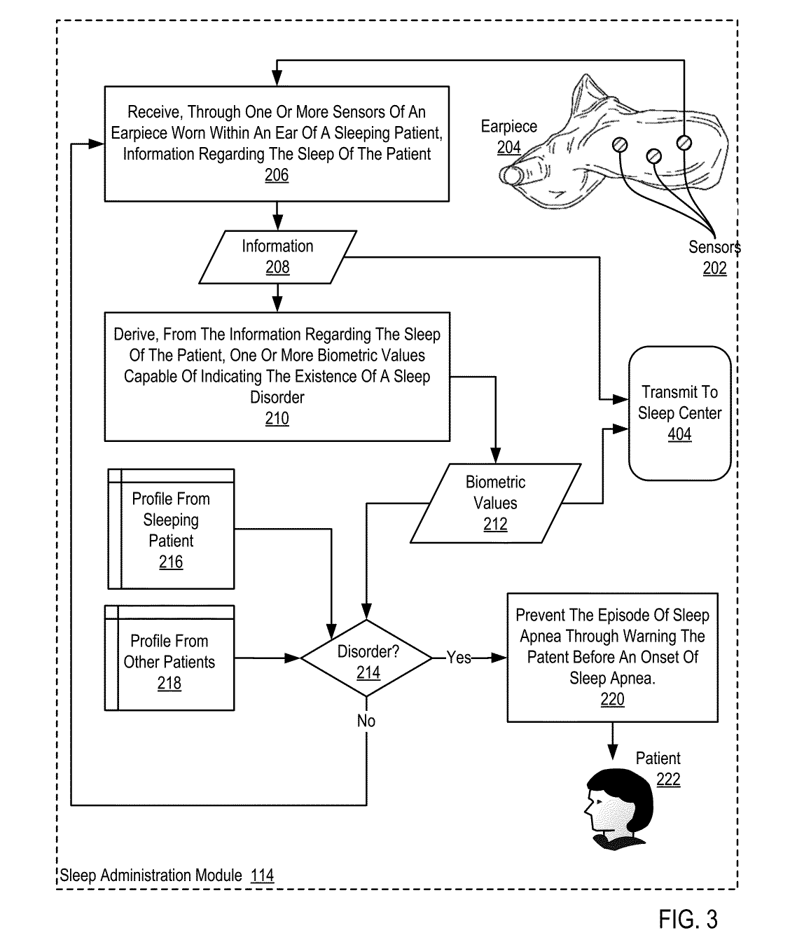

For further explanation, FIG. 3 sets forth a flow chart illustrating an example method of administering a sleep disorder. The method of FIG. 3 includes receiving (206), in a sleep administration module (114) through one or more sensors (202) of an earpiece (204) worn within an ear of a sleeping patient (222), information (208) regarding the sleep of the patient.

The example method of FIG. 3 also includes deriving (210), by the sleep administration module (114) from the information (208) regarding the sleep of the patient (222), one or more biometric values (212) capable of indicating the existence of a sleep disorder. In the example of FIG. 3, the sensed information (208) can include electroencephalography, electromyography, electrooculography, electrocardiography, accelerometry, reflective pulse oximetry, audio, temperature, and other sensed information as may occur to those of skill in the art. In the example of FIG. 3, the biometric values (212) can include pulse rate, body temperature, blood oxygen level, rapid eye movement sleep, non-rapid eye movement sleep, snoring, blood pressure, muscle tension, and other values derived from sensed information as may occur to those of skill in the art. The method of FIG. 3 also includes an optional step of transmitting (404), by the sleep administration module (114), the sensed information (208) and/or the biometric values (212) to a sleep center (224 on FIG. 1).

The example of FIG. 3 includes determining (214), by the sleep administration module (114), whether the one or more biometric values (212) indicate that the sleeping patient (222) is presently experiencing a sleep disorder. Examples of sleep disorders administrable by the example method of FIG. 3 include sleep hypopnea, sleep apnea, and a sleep disorder that is a precursor to an episode of sleep apnea. The method of FIG. 3 includes preventing (220), by the sleep administration module (114), an episode of sleep apnea through warning the patient (222) before an onset of sleep apnea when it is determined (214) that a precursor condition is present.

The method of FIG. 3 includes sleep administration module (114) is configured to prevent an episode of sleep apnea through a warning (120) to the patient before an onset of sleep apnea when the sleep administration module determines that a precursor state is present. Examples of warnings include audible tones provided through a speaker or earphone on the earpiece, a vibration warning through a buzzer or the like, integrated within the earpiece, a prerecorded voice warning, and so on.

In the method of FIG. 3, determining (214) whether the biometric values (212) indicate that the sleeping patient is presently experiencing a sleep disorder can be carried out by comparing (228) the biometric values (212) with a predetermined sleep disorder profile (216) personalized for the sleeping patient. Also in the method of FIG. 3, determining (214) whether the biometric values (212) indicate that the sleeping patient is presently experiencing a sleep disorder can alternatively be carried out by comparing (230) the one or more biometric values (212) with a predetermined sleep disorder profile (218) derived from sleep disorder data of a number of other patients.

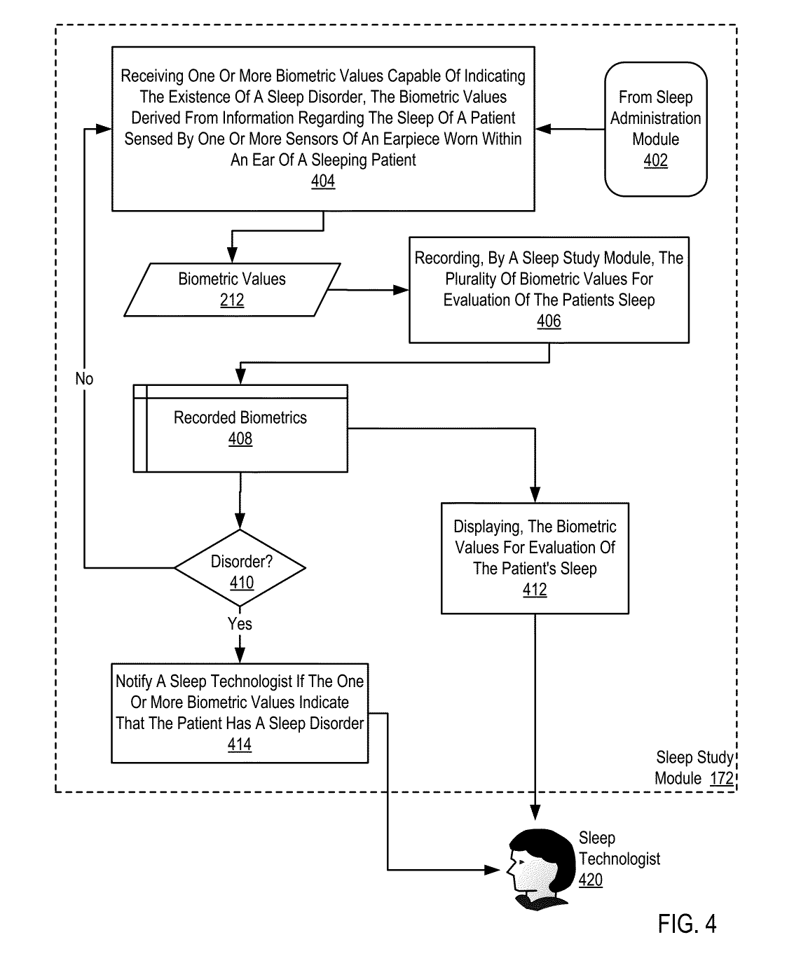

For further explanation, FIG. 4 sets forth a flow chart illustrating an example method of sleep study. The method of FIG. 4 includes receiving (404), by a sleep study module (172), one or more biometric values (212) capable of indicating the existence of a sleep disorder. The biometric values (212) are derived from information regarding the sleep of a patient sensed by one or more sensors of an earpiece worn within an ear of a sleeping patient. The example method of FIG. 4 also includes recording (406), by the sleep study module (172), the biometric values (408) for use by a sleep technologist in evaluating the patient's sleep.

The method of FIG. 4 also includes displaying (412), by the sleep study module (172), the biometric values (408) for evaluation of the patient's sleep. That is, the sleep study module displays the biometric values on a computer screen, printer output, or the like, for review or evaluation by a sleep technologist (420). The method of FIG. 4 also includes determining (410), by the sleep study module (172), that is, under automation, whether the one or more biometric values indicate that the patient has a sleep disorder. The method of FIG. 4 also includes notifying (414), by the sleep study module (172), a sleep technologist (420) if the one or more biometric values (408) indicate that the patient has a sleep disorder.

Sleep study is carried out generally in embodiments by use of electroencephalography (‘EEG’), electromyography (‘EMG’) and electrooculography (‘EOG’) information from sensors on an earpiece within the ear. The stage of sleep typically is taken from EEG and EMG information, from measures of the power of signals at certain frequencies. Stage 2 sleep will give sudden, short high-voltage wave bursts occurring at 12-14 Hz. Stage 3 sleep will show theta (4-7 Hz) and delta waves (1-4 Hz) with skeletal muscles very relaxed. Stage 4 is “slow wave sleep” because of delta waves, with a body turn approximately every 20 minutes. Rapid eye movement (‘REM’) sleep is indicated after the first four stages when frequency goes back to alpha waves, body temperature increases, heart rate increases, respiratory rate increases, blood pressure increases, the brain uses even more oxygen than when awake, eyes move rapidly. This particular signal from eye movement may be classified as EMG rather than EOG and is easily detected with information from the earpiece sensors.

Regarding REM sleep, sleep alternates between REM and non-REM or NREM; REM occurs about every 90 minutes and increases in length from 5-10 minutes to 20-50 minutes. The amount of REM sleep typically is determined in embodiments from sensor information by detecting eye movement using EOG and EMG. A person feels most rested when awakened just after a REM cycle, so that warnings can signal a person to awaken when apparatus in embodiments detects that REM is finished.

Clenching and grinding of teeth is detected in embodiments by use of EMG. For each skeletal muscle, there is an optimal longitudinal length at which the maximum muscle activation can occur; muscle activation of the muscles of mastication can be measured using EMG. When placing a sleep disorder appliance into the mouth, the teeth become separated, slightly lengthening the muscles of mastication, preventing the electrical signal from the muscles of mastication from being as intense as having no teeth separation. For a patient that is prone to clenching, the clenching intensity will be decreased when wearing the oral appliance. Warnings to the patient in embodiments effectively implements relaxation training Some embodiments play music or tones only when a patient is relaxed (or vice versa) using EMG detection of nearby muscle activity (muscles of mastication).

Accelerometry from within the ear includes in embodiments nine degrees of freedom (9 DOF accelerometry). 9 DOF accelerometry includes multiple axes of detection from which, based on acceleration due to gravity, a patient's resting head position can be determined. Then embodiments can alert the patient to changes into nonoptimal sleep positions.

Oximetry typically is implemented as reflection pulse oximetry from within the ear or transmission pulse oximetry around the pinna. Embodiments can use both red (600-750 nm) and infrared light (850-1000 nm) to illuminate blood and use a photosensor to measure either transmission or reflection. Red light at 660 nm reflects off of hemoglobin when it is saturated (HbO2) and infrared light at 940 nm reflects off of de-oxygenated hemoglobin (Hb).

$$Ratio of Ratios \sim \frac{ln ( \frac{{Red}_{systole}}{{Red}_{diastole}} )}{ln ( \frac{{IR}_{systol}}{{IR}_{diastole}} )} \qquad Formula 1$$

The ‘ratio of ratios’ according to Formula 1 is calibrated in embodiments to determine peripheral capillary oxygen saturation or SpO₂ in percentage, using a lookup table to determine the actual percentage. SpO₂ (%) can be measured, a value that decreases during an apneic episode. Pulse rate (beats per minute) can be measured in embodiments with oximetry because there is variable light absorption due to pulsatile volume of arterial blood. When measuring from within the ear canal, direct reflective pulse oximetry towards the superficial temporal artery, which runs anterior to the canal, or associated vasculature. When measuring in locations requiring light transmission detection (instead of reflection), such as through the pinna or ear lobe, embodiments use a clip that places lights on one side of tissue and photosensor on the other side. While using an oral appliance for obstructive sleep apnea, there are no acute decreases in oxygen saturation unless sleep apnea occurs via central sleep apnea where there is no respiratory effort by the patient. Embodiments therefore can alert a patient when oxygen saturation decreases below a threshold.

Sensors in embodiments can include a microphone to sense or record snoring sounds. Snoring sounds decrease with use of an obstructive sleep apnea oral appliance. Snoring sounds can also be used to indicate oral appliance (mandibular advancement appliance) effectiveness at maintaining pharyngeal patency. Audio from snoring in embodiments can complement accelerometer information to determine patient movements during sleep, alerting a patient to change positions when snoring indicates nonoptimal body position.

Additional warning-type technology in embodiments can include a speaker or earphone integrated in the earpiece that delivers information directly into a patient's ear without disrupting others nearby. Audible warnings can include alerts to change sleeping position, alerts to wake a patient, music or relaxation sounds, including playing slow breathing sounds for breath matching, to aid a patient in falling asleep. These alerts and sounds in embodiments are implemented with a phone paired via Bluetooth with a source of soothing sounds or music and, in some embodiments, are supportive of sleep-related training such as EMG relaxation training

An embodiment includes a Piezo sensor to detect pulse from within the ear. This is in addition to pulse oximetry which in some embodiments may have too low measurement/calculation frequency or too low noise for pulse detection. A Piezo sensor is mounted on the earpiece so as to contact skin in the ear canal and detect pulse through impulses affecting skin pressure on the sensor. In at least one embodiment, skin pressure noise from snoring, movement, and the like, is canceled with audio noise from a microphone.

In some embodiments, earpiece sensors can include one or more active in-ear readers for sensors mounted on an oral appliance and directed to sleep disorder appliance compliance, including a passive RadioFrequency Identification (RFID) tag, a Near Field Communications (‘NFC’) tag, a contactless smart card, or the like, attached to the oral appliance and registered with the active in-ear reader in an earpiece when in use to determine appliance compliance. A passive tag in an embodiment is switched on only when the oral appliance is locked into the patient's mouth, working only when two pieces of the RFID tag are connected to each other via electrodes to the gums. One part of such an RFID tag is attached to the patient, making electrical contact to a second part of the RFID tag mounted on the oral appliance only when the appliance is worn. In another embodiment, a passive RFID tag is split into two parts as an open circuit, and the act of placing the oral appliance in the mouth and pressing it onto the teeth mechanically connects the two for further operation with an active RFID reader.

The active in-ear reader in the earpiece sends an RF signal to power a passive RFID or NFC tag installed on the oral appliance. The active in-ear reader can send an RF signal that powers a passive tag on the oral appliance, with the passive tag connected to one or more physiological sensors, temperature, O2, pressure against teeth, electrical conduction, and so on, with the sensor data then sent back to the active reader in the ear. A force sensor may be embedded in an oral appliance to be pressed against tooth during use, with force data be transferred to the in-ear reader to determine appliance compliance. A temperature sensor may be embedded into an oral appliance, with temperature data transferred to the in-ear reader to determine appliance compliance.

In some embodiments, an oral appliance contains a piezo or bone conduction transducer, with audio vibrations received by microphone in the ear or on the appliance, with a connection to the earpiece by RFID, ultrasound, vibration, and so on. An ultrasound signal in such embodiments is sent from the ear device through the body and makes contact with the oral appliance. The signal is then passively modulated and reflected through the body and back to the ear device. The modified signal received by the ear device confirms proper placement of the oral appliance in the mouth.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. A method of sleep study, the method comprising:
receiving, by a sleep study module, one or more biometric values capable of indicating the existence of a sleep disorder, the biometric values derived from information regarding the sleep of a patient sensed by one or more sensors of an earpiece worn within an ear of a sleeping patient; and
recording, by the sleep study module, the plurality of biometric values for evaluation of the patient's sleep.

2. The method of claim 1 further comprising displaying, by the sleep study module, the biometric values for evaluation of the patient's sleep.

3. The method of claim 1 further comprising determining, by the sleep study module, whether the one or more biometric values indicate that the patient has a sleep disorder.

4. The method of claim 3 further comprising notifying, by the sleep study module, a sleep technologist if the one or more biometric values indicate that the patient has a sleep disorder.

5. The method of claim 1 further comprising:
receiving, in a sleep administration module through one or more sensors of an earpiece worn within an ear of a sleeping patient, information regarding the sleep of the patient;
deriving, by the sleep administration module from the information regarding the sleep of the patient, one or more biometric values capable of indicating the existence of a sleep disorder; and
transmitting, by the sleep administration module to the sleep study module, the biometric values for evaluation.

6. The method of claim 1 wherein the biometric values comprise pulse rate, body temperature, blood oxygen level, rapid eye movement sleep, non-rapid eye movement sleep, snoring, blood pressure, and muscle tension.

7. The method of claim 1 wherein the sleep study module resides in a sleep center.

8. The method of claim 1 wherein the biometric values derived from information regarding the sleep of a patient sensed by one or more sensors of an earpiece worn within an ear of a sleeping patient are received from a sleep administration module residing in the patient's home.

9. A system of sleep study, the system comprising a computer processor, a computer memory operatively coupled to the computer processor, the computer memory having disposed within it computer program instructions that, when executed by the computer processor, cause apparatus of the system to function by:
receiving one or more biometric values capable of indicating the existence of a sleep disorder, the biometric values derived from information regarding the sleep of a patient sensed by one or more sensors of an earpiece worn within an ear of a sleeping patient; and
recording the plurality of biometric values for evaluation of the patient's sleep.

10. The system of claim 9 wherein the computer memory also has disposed within it computer program instructions that cause apparatus of the system to function by displaying the biometric values for evaluation of the patient's sleep.

11. The system of claim 9 wherein the computer memory also has disposed within it computer program instructions that cause apparatus of the system to function by determining whether the one or more biometric values indicate that the patient has a sleep disorder.

12. The system of claim 9 wherein the computer memory also has disposed within it computer program instructions that cause apparatus of the system to function by notifying a sleep technologist if the one or more biometric values indicate that the patient has a sleep disorder.

13. The system of claim 9 further comprising a sleep administration module including a computer processor, a computer memory operatively coupled to the computer processor, the computer memory having disposed within it computer program instructions that, when executed by the computer processor, cause apparatus of the system to function by:
receiving, through one or more sensors of an earpiece worn within an ear of a sleeping patient, information regarding the sleep of the patient;
deriving, from the information regarding the sleep of the patient, one or more biometric values capable of indicating the existence of a sleep disorder; and
transmitting, to the sleep study module, the biometric values for evaluation.

14. The system of claim 9 wherein the biometric values comprise pulse rate, body temperature, blood oxygen level, rapid eye movement sleep, non-rapid eye movement sleep, snoring, blood pressure, and muscle tension.

15. The system of claim 9 wherein the sleep study module (172) resides in a sleep center.

16. The system of claim 9 wherein the biometric values derived from information regarding the sleep of a patient sensed by one or more sensors of an earpiece worn within an ear of a sleeping patient are received from a sleep administration module residing in the patient's home.
